(19) United States
(12) Patent Application Publication
(10) Pub. No.: US 2018/0296383 A1
(43) Pub. Date: Oct. 18, 2018

(54) Method To Promote Health And Function of The Penis And Vagina Utilizing A Combination of Sound Waves, Electrical Stimulation, Light, and Vacuum

(71) Applicants: RAYMOND R BLANCHE, CHATHAM, NJ (US); MICHAEL D. MOSK, OSWEGO, IL (US)

(72) Inventors: RAYMOND R BLANCHE, CHATHAM, NJ (US); MICHAEL D. MOSK, OSWEGO, IL (US)

(21) Appl. No.: 15/953,267

(22) Filed: Apr. 13, 2018

Related U.S. Application Data
(60) Provisional application No. 62/485,046, filed on Apr. 13, 2017.

(51) Int. Cl. A61F 5/41; A61N 5/06; A61N 1/36

(52) U.S. Cl. CPC A61F 5/41; A61N 5/0622; A61N 1/36107; A61F 2005/417; A61F 2005/418; A61N 2005/0611; A61F 2005/412

(57) ABSTRACT

This relates to a device that combines ultrasound energy, electrical energy, light, and vacuum to improve the general health function of the penis and the vagina. It is intended to be safe, easy to use way to increase and improve the vascular supply to the penis and vagina. A system will comprise a power source, vacuum pump and a stimulatory device. The male device comprises a tubular receptacle including an inner tube and an outer tube; the inner tube having electrodes, ultrasound transducers, and light sources, all attached to the inner tube to direct their energy to the inside, the inner tube positioned within the outer tube. The front is closed by a cup that fits on and receives the outer tube rim. The back is closed by a rear base assembly that receives an end of the inner tube so the inner tube does not contact the outer tube.

### CROSS-REFERENCE TO RELATED APPLICATIONS

This application is a nonprovisional application that claims the benefit of U.S. Application Ser. No. 62/485,046, filed 13 Apr. 2017 by these inventors, and whose contents are incorporated by reference herein in their entirety.

### FIELD OF THE EMBODIMENTS

The field of the embodiments of the present invention relate to an apparatus and method for combining ultrasound energy, electrical stimulation, light, and vacuum for the promotion of health and function of the penis and/or vagina by stimulation of blood flow and sensory nerves.

### BACKGROUND OF THE EMBODIMENTS

The most common approach to treating sexual dysfunction is treatment with prescription or over the counter (OTC) drugs. This is typically done due to the ease of use and the overall rate of success with a majority of the men using such drugs (e.g. VIAGRA®, registered trademark of Pfizer, Inc., NY for a sildenafil citrate compound for treating erectile dysfunction). However, as with all systemic drugs, there are side effects including but not limited to general drug intolerances, nausea, low blood pressure, etc. Injections directly into the penis can have severe side results from extensive use and can cause harmful effects after only a single use. In turn, some seek out vacuum-based devices. These devices are purely mechanical and eliminate the aforementioned problems of invasive approaches, but when used with “excessive” settings can further be problematic. Most recently, however, high peak power pulsed ultrasound has been used to create micro-wounds in tissue to stimulate new tissue growth. Thus, there is a need of a device and methodology that incorporates ultrasound into a health device for the genitalia and removes the potentially dangerous side effects of the above described treatments.

Various devices and methodologies are known in the art. Some devices previously used to treat male erectile dysfunction have employed the use of high vacuum to effect erections; however, tissue damage to the area was often a side-effect of such treatment, and these devices are now in disfavor.

There are numerous other disorders that affect the health and function of the penis and vagina other than sexual dysfunction, and are frequently associated with pain. Peyronie's disease affects the penis, and is characterized by a curvature of the organ when erect due to the build up of scar tissue within the tunica albuginea, a thick elastic membrane. Peyronie's disease can lead to pain, as well as erectile dysfunction. Disorders of the bladder or prostate, and the overall health of the nerves of the urogenital tract can also be included within this group. Many of these disorders are considered part of the group known as Lower Urinary Tract Symptoms (“LUTS”), and affect both men and women.

Women are affected by a number of conditions, such a decreased quantity of vaginal secretions, nerve problems, or increased urinary tract infection incidence. Other issues include vaginal wall atrophy due to lack of estrogen, and reduced collagen and elastin production by post-menopausal women. Vulvodynia is a painful disorder sometimes described as an intense burning. Severe cases that can't be effectively treated by medications, for example, local anesthetics or exercise or biofeedback are surgically treated by removal of the affected tissue.

Other devices utilize various frequencies of light, and yet others use ultrasound. However, their structure and means of operation are substantially different from the present disclosure. The other inventions also fail to solve all the problems taught by the present disclosure. Embodiments of the present invention combine the use of vacuum, ultrasound treatment and light exposure into a single device that could be used to treat a variety of medical conditions. A pending U.S. patent application (Ser. No. 15/497,697) by the present inventors describes treating cellulite with this combination of therapies. Embodiments of the present invention are intended for use in treating the general health of the male penile and female vaginal areas. At least one embodiment of the present invention is presented in the drawings below and will be described in more detail herein.

### SUMMARY OF THE EMBODIMENTS

The embodiments of the present invention teach and describe a method for combing ultrasound energy, electrical energy, and vacuum to treat the health and improve the function of the penis as well as the vagina. Its inherent concept and design is intended to be safe, easy to use and represents a natural way to increase and improve the vascular supply to the penis and vagina. Further, these improved results will be achieved in a more lasting way without and hazards of systemic drugs and their transient nature or tissue damaging properties.

In one embodiment of the present invention, intended for use by males, there is a device comprising:
a tubular receptacle including an inner tube and an outer tube;
the outer tube having a first end that terminates in a rim, and a second end that terminates in a flange;
the inner tube comprising a plurality of perforations therethrough, a pair of electrodes, a plurality of ultrasound transducers, and a plurality of light sources, all attached to the surface of the inner tube and directing their energy to the inside of the inner tube; the inner tube positioned within the outer tube;
a cup, the cup sized to fit on and receiving the outer tube rim, the outer tube rim retaining the inner tube therein;
a rear base assembly, the rear base assembly comprising a base, the base including an inner ring, the inner ring sized to receive and receiving a gasket and an end of the inner tube, retaining the inner tube so the inner tube does not contact the outer tube;
a connection for a vacuum source; and
the light sources provide light at a wavelength ranging from about 660 nm to about 940 nm.
The system includes a power source and a source of a vacuum, such as a vacuum pump.

In an embodiment for use by females, the device comprises a handle, the handle comprising a handle member, a guard attached to the handle member, the guard having an forward surface sized to receive, and receiving a flange therein, and a handle cap, the flange being positioned between the guard and the handle cap. The device includes a perforated inner tube onto which is positioned a pair of electrodes to provide electrical stimulation. A plurality of Light Emitting Diodes (“LEDs”) are within the female device, and provide light at a wavelength that ranges from about 660 nm to about 940 nm. The handle includes a connection to a source of vacuum. The inner tube is positioned within an outer tube element, the outer tube element including a plurality of ultrasound transducers attached thereto. The outer tube and female device is closed by a cap attached to a rim on the outer tube, and the handle components close off the rear of the device. In use, the outside of the female device is covered with a quantity of ultrasound gel, and inserted within the vagina. When turned on, the device provides a combination of vacuum, ultrasound, electrical and light stimulation to the affected area, in a pulsed or continuous cycle.

### BRIEF DESCRIPTION OF THE DRAWINGS

FIG. 1 is a perspective view of a male embodiment of the system of the present invention.

FIG. 2a is a side perspective view of the male embodiment of the present invention.

FIG. 2b is a front perspective view of the male embodiment of the present invention.

FIG. 3 is a cross-sectional view of the embodiment of FIG. 2b, taken along line 3-3.

FIG. 4 is a longitudinal cross section view of the embodiment of FIG. 2, when rotated 90 degrees.

FIG. 5 is a longitudinal cross section view of the embodiment of FIG. 2.

FIG. 6 is an exploded view of the male embodiment shown in FIG. 2.

FIG. 7 is a perspective view of a female embodiment of the present invention.

FIG. 8 is a rear perspective view of the female embodiment of FIG. 7.

FIG. 9 is a side view of the female embodiment of FIG. 7.

FIG. 10 is a top view of the female embodiment of FIG. 7.

FIG. 11 is an exploded, frontal, view of the female embodiment shown in FIG. 7.

FIG. 12 is an exploded, rear, view of the female embodiment shown in FIG. 7.

FIG. 13 is a longitudinal sectional view of the female embodiment.

FIG. 14 is a longitudinal cross section view of the female embodiment.

FIG. 15 is a front view showing the female embodiment in use with a female user.

### DETAILED DESCRIPTION OF THE INVENTION

Embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

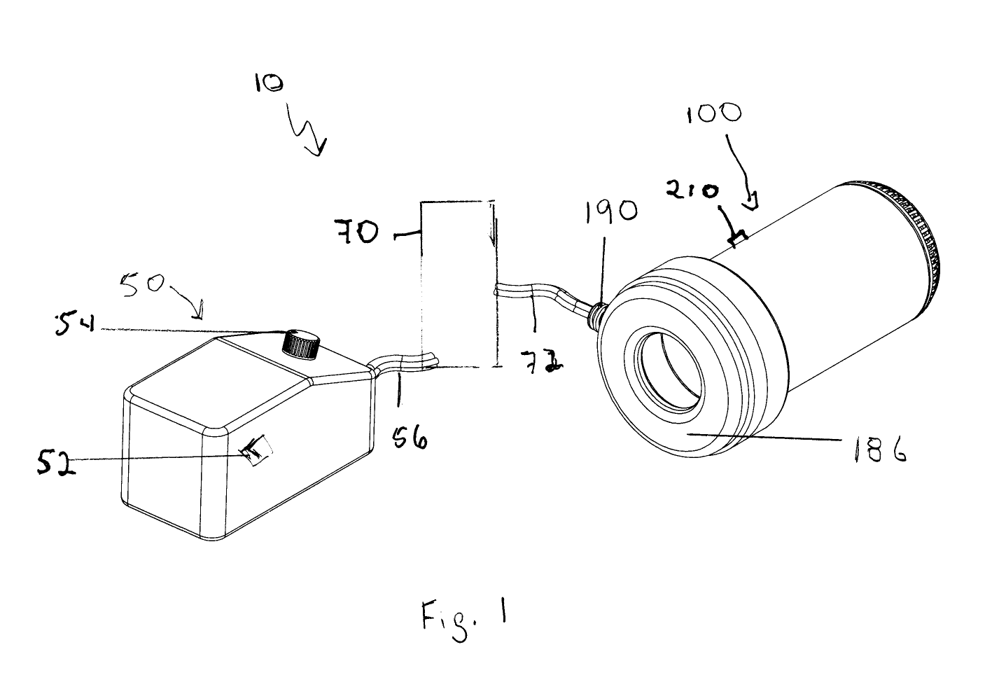

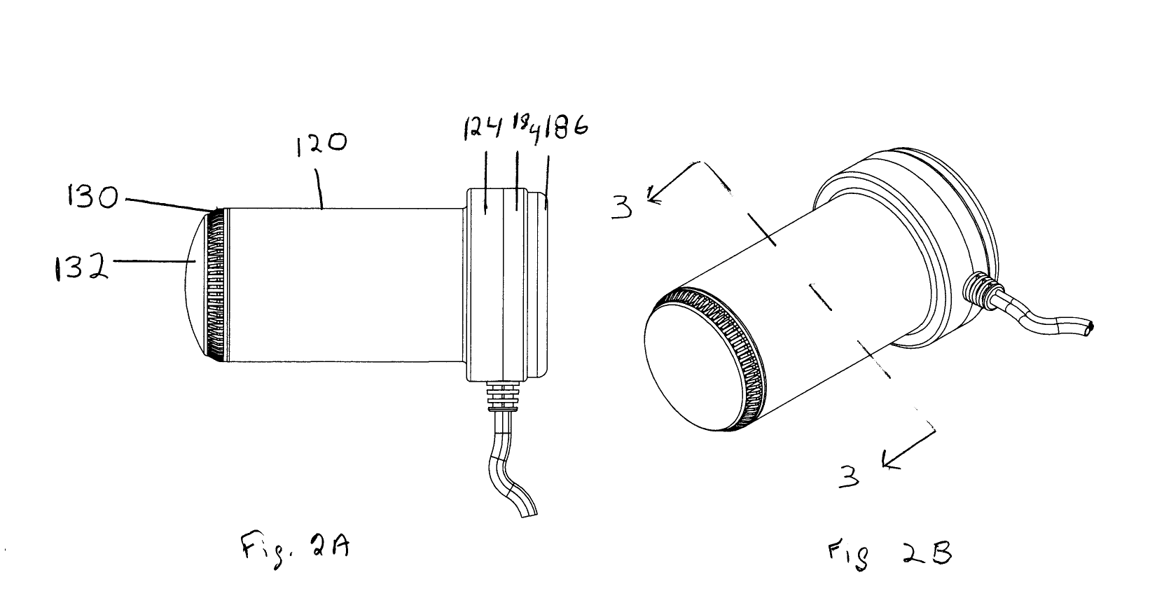

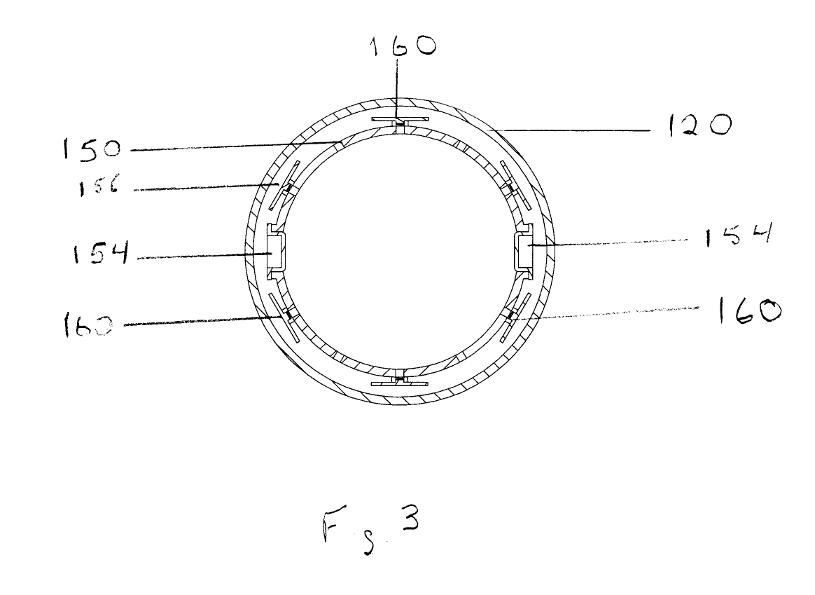

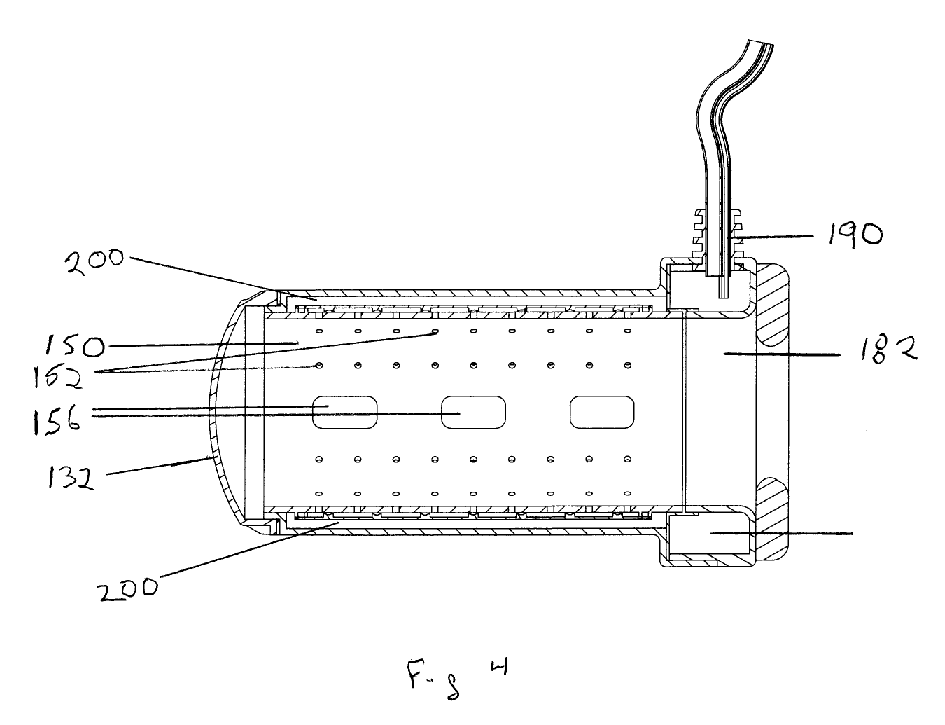

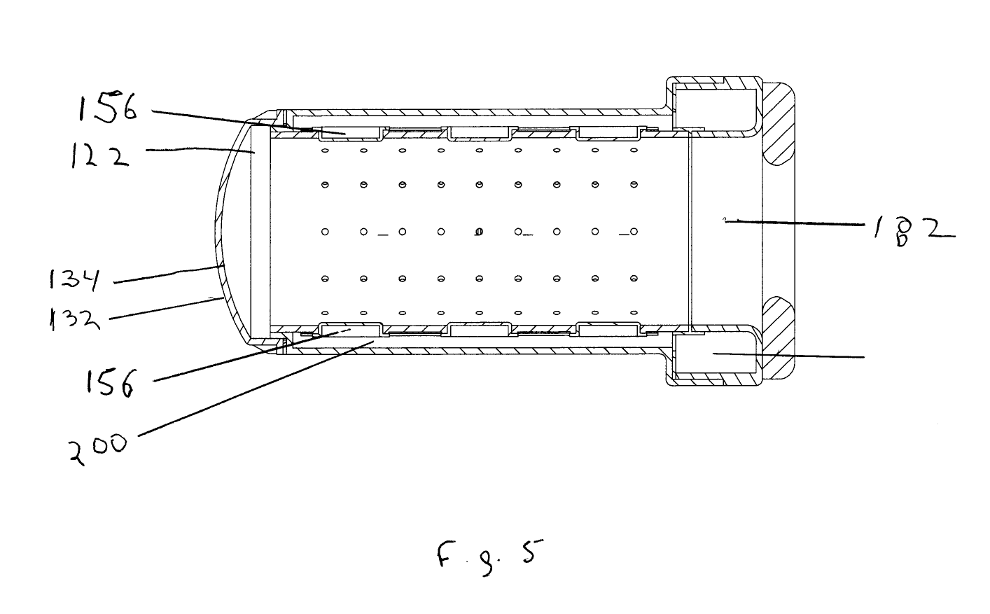

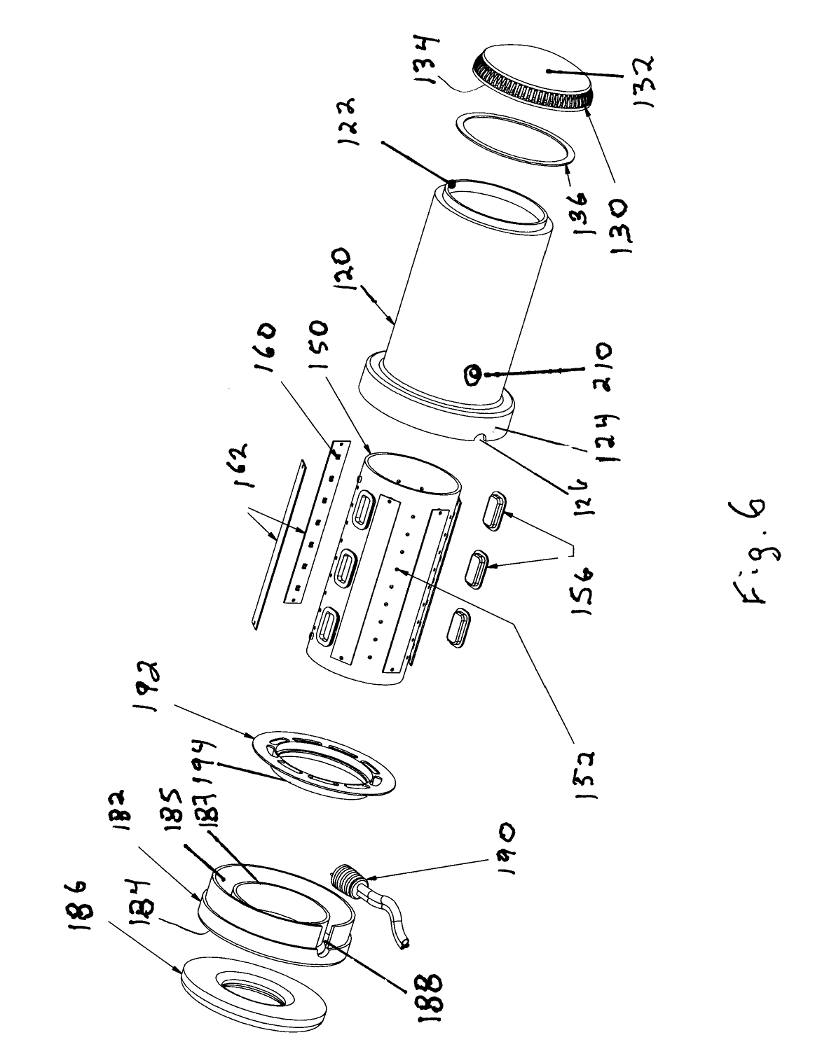

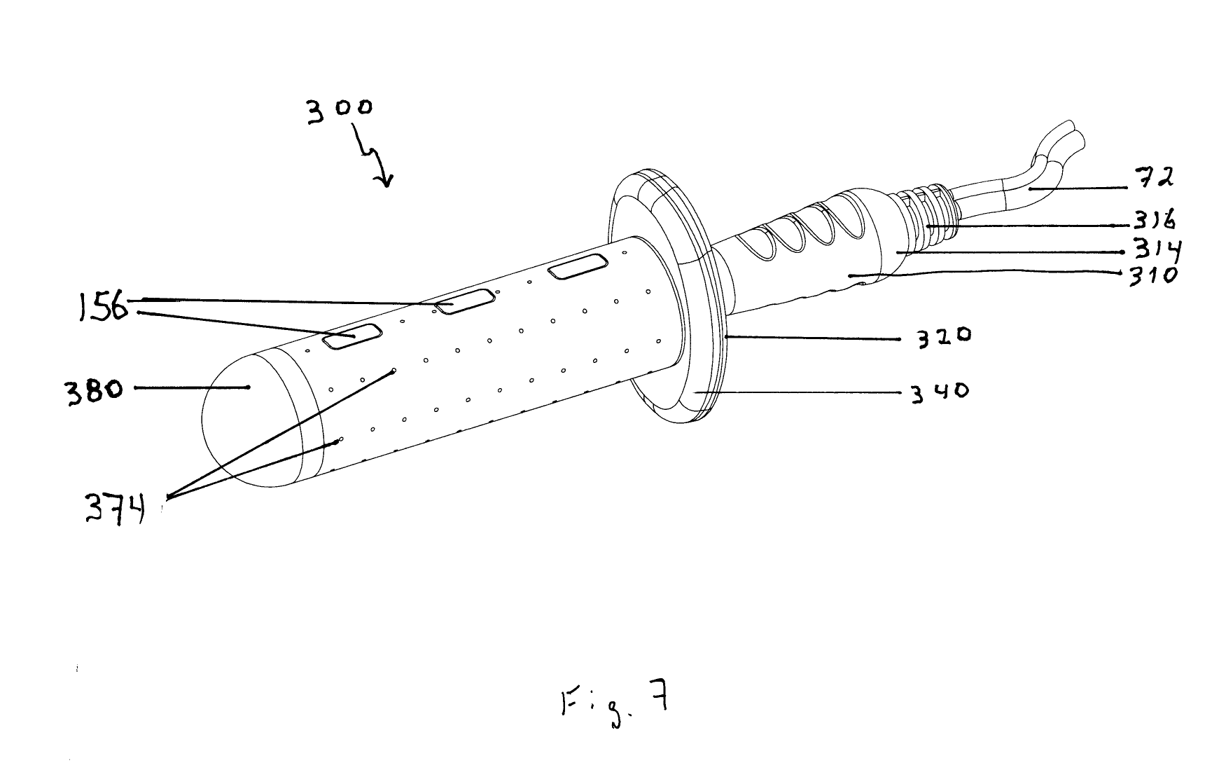

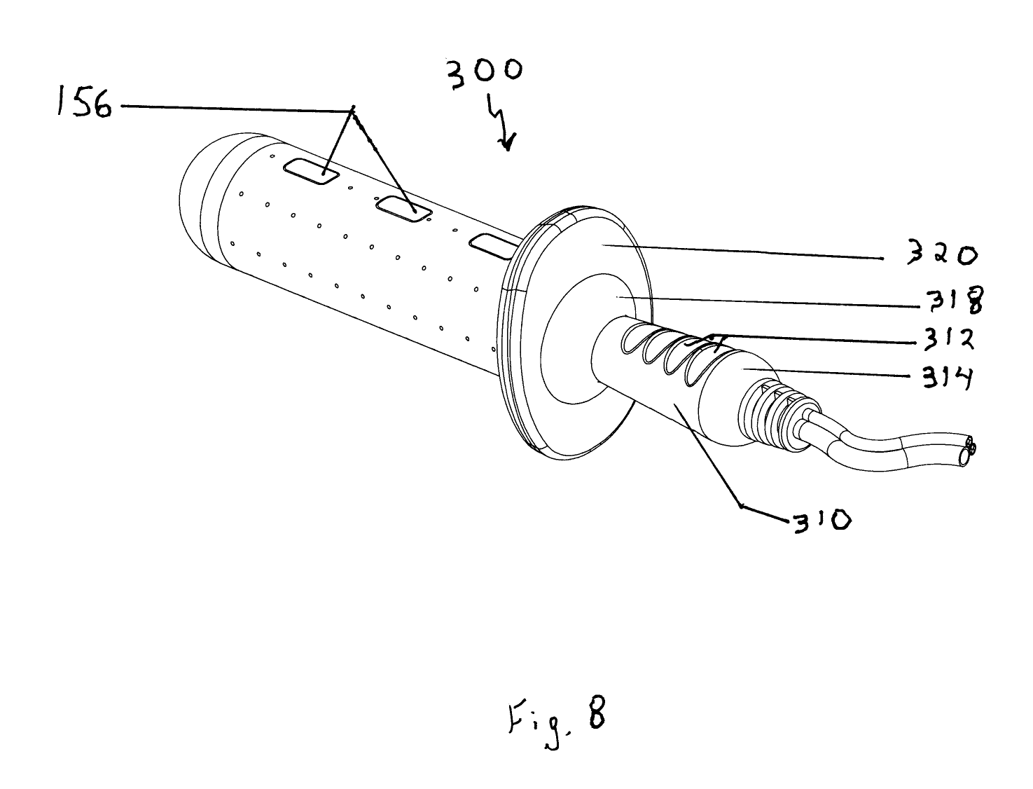

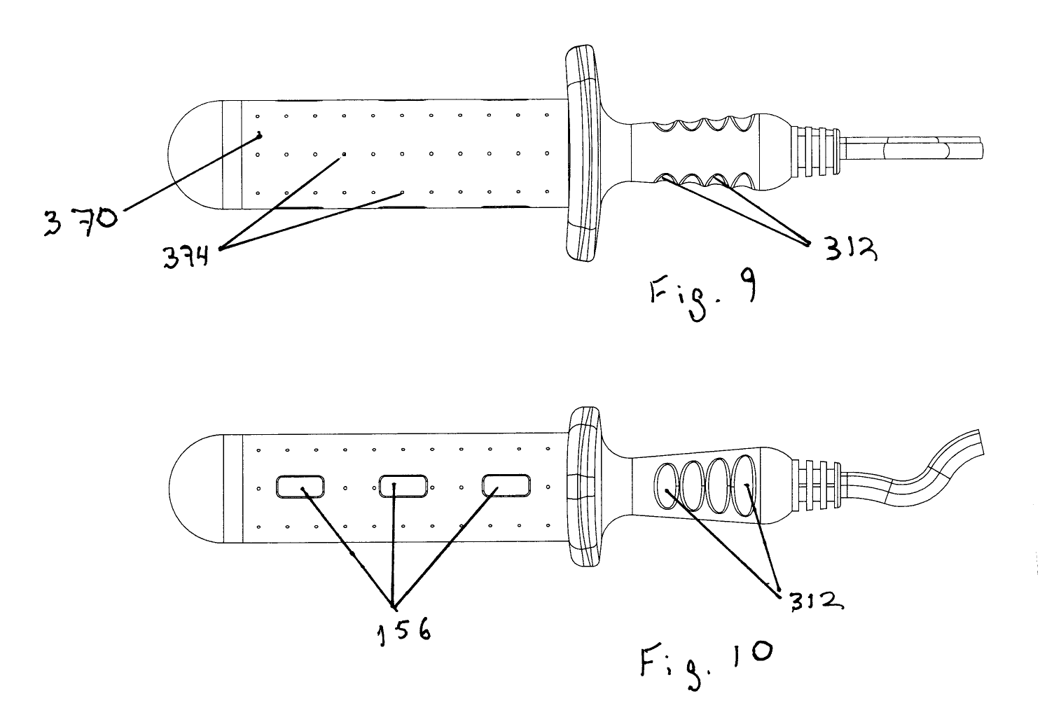

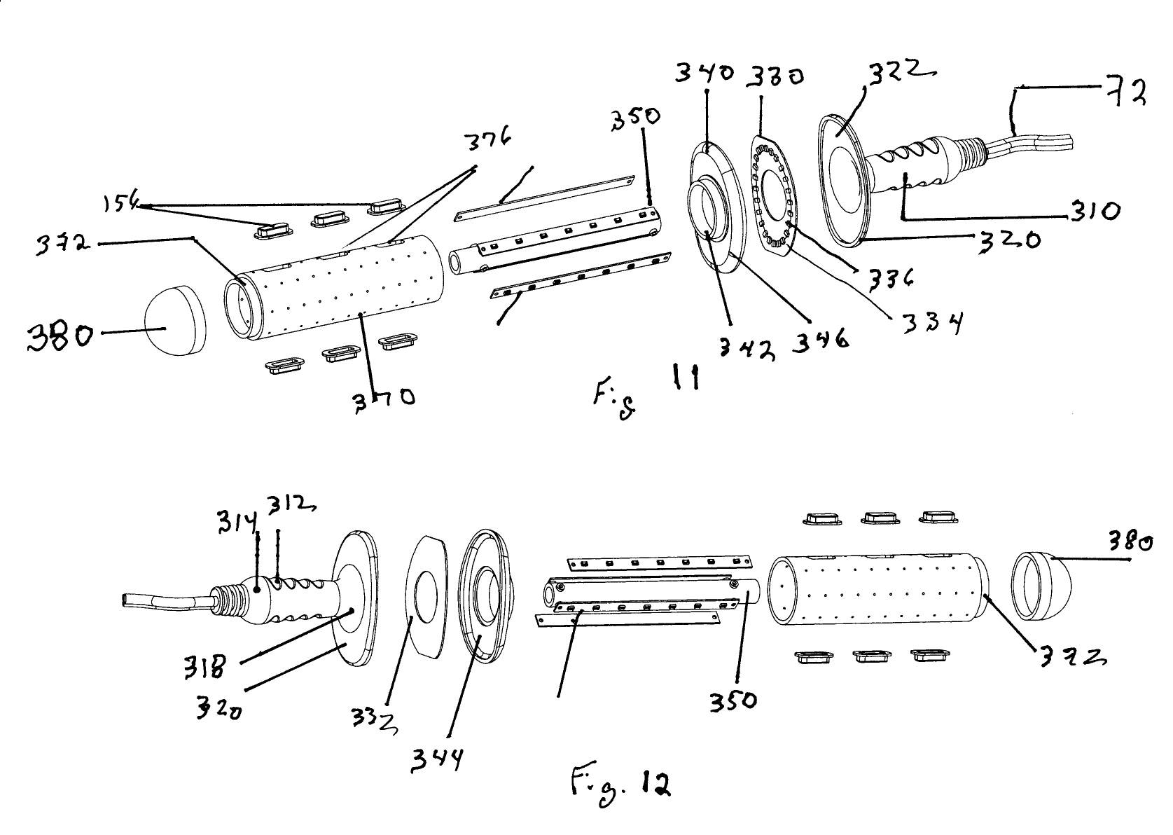

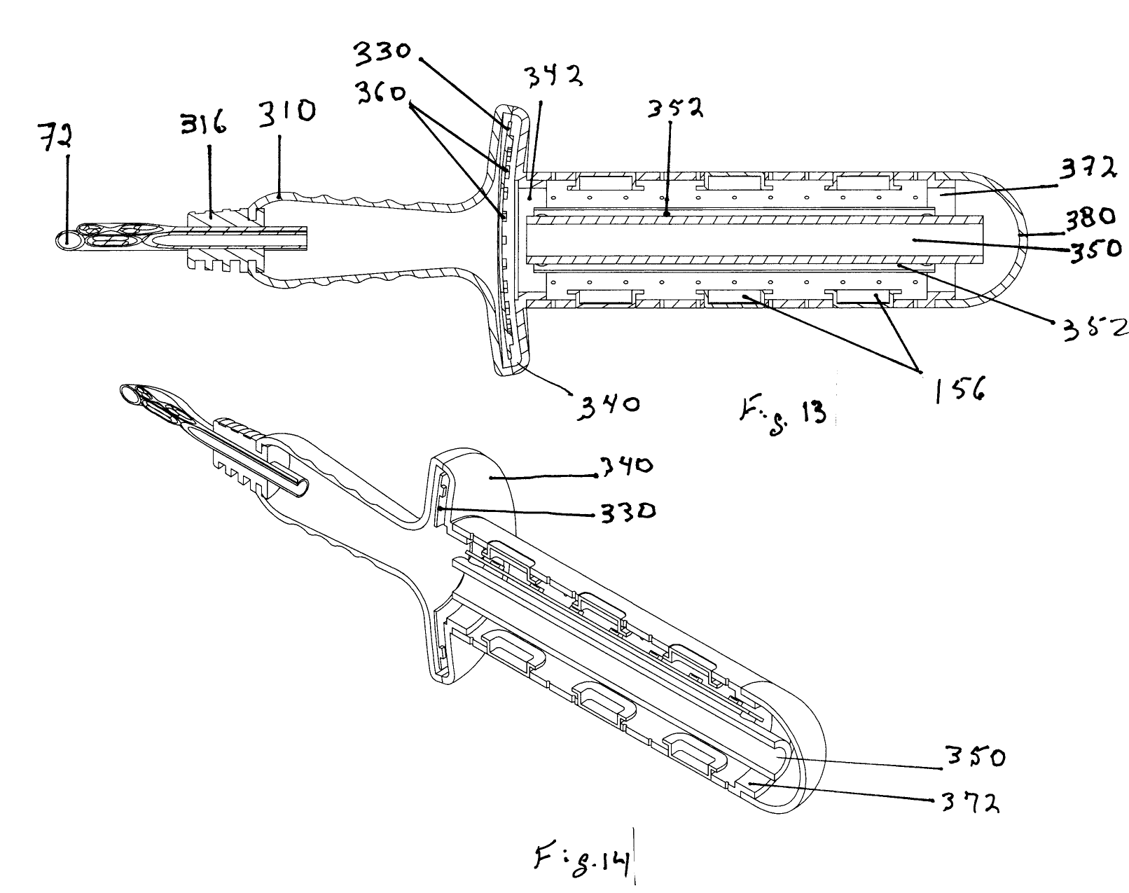

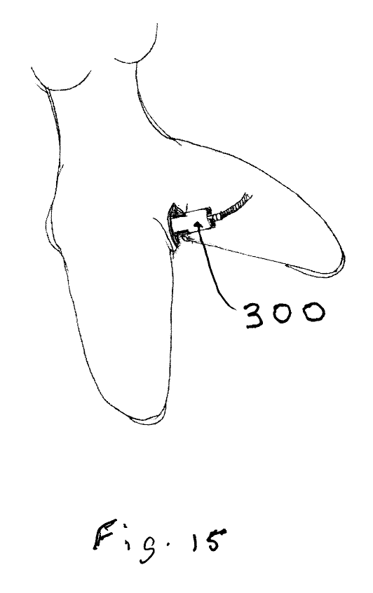

FIGS. 1-6 illustrate an embodiment of the present invention 100 intended to be used by males. The apparatus combines ultrasound energy, electrical stimulation, light, and vacuum for the promotion of health and function of the penis by stimulation of blood flow and sensory nerves. Initially, this will provide improved erectile function with the eventual goal of more permanent vascularization and improved function. Another embodiment of this device, with slight positioning modifications to the modalities, may be used in and on the vagina (FIG. 7) for improved sensory stimulation and increased moisturization of the vaginal area. This is accomplished by stimulating the more sensitive lower one third of the vagina and the Bartholin gland and duct which secretes fluid for lubrication. The other oil and fluid secreting glands, for example, the Skene, sweat and sebaceous glands, and the cervical mucosa, can also be affected by the stimulatory device of the present invention.

One purpose of the present application is to provide a safe and effective method to treat without systemic drugs, injections or potentially damaging vacuum type devices. This process emphasizes specificity of treatment by only effectively treating the vascular supply to the area. Both ultrasound, light and electrical energy have the ability to penetrate deeply into tissue to increase the vascular supply. Specific pre-set parameters of all the energy forms ensure maximum benefit with minimal chance for tissue damage.

An embodiment of the stimulatory system is shown as reference numeral 10 in FIG. 1, and comprises a power source 50 and a stimulatory device 100 which are in communication with a vacuum pump 70. Power source 50 includes an on-off switch 52 and a controller 54 to adjust the vacuum; a power cord is not shown. The controller 54 can be a rheostat, pressure gauge, or other means of controlling the vacuum that will be applied to the stimulatory device. The power source 50 is in electrical communication with the vacuum pump 70 by a power cord 56, and the vacuum pump 70 is in electrical communication with, and the vacuum connected to, the stimulatory device by a combination cable 72. The power source 50 may also include a microprocessor, which can be programmed, either directly or through a wireless communication means, to provide a range of cycles for the vacuum, the electrical stimulation, the ultrasound transducers, and the light sources. An embodiment of the stimulatory device for males has reference numeral 100, and an embodiment of the stimulatory device for females has reference numeral 300.

Male stimulatory device 100 comprises an outer body tube 120 and an inner body tube 150. Outer body tube 120 is a circular tube having an outer rim 122 at the tube's front end, and whose back end comprises a circular flange 124 that includes a notch 126 on portion of the flange 124. The front end of outer body tube 120 is closed by a front cup 130 that contains an outer side 132 and an inner side 134. The inner side 134 is sized to receive, and receives the outer rim 122 by means of a pressfit. A gasket 136 is positioned on outer rim 122 and between the front cup 130 and provides a seal between the outer body tube 120 and the front cup 130. In alternate embodiments, the front cup could be attached to the outer rim by being screwed onto threads (not shown) formed on the outer rim.

As shown in FIGS. 3-6, the inner body tube 150 is a circular tube sized to fit within the outer body tube 120, and as seen in FIG. 3, will not be in contact with the inside walls of the outer body tube. The inner body tube 150 will comprise a cylindrical tube that will accommodate varying sized penises. Inner body tube 150 will be perforated (FIG. 6) and the perforations 152 provide openings for the flow of air out of, and into, the stimulatory device when vacuum is applied to, or let out of, the stimulatory device 100. A set of electrodes 154 will be mounted on the surface of the inner body tube 150, as shown in FIG. 3. The electrodes 154 are metal strips that will act as the active and dispersive pad for the delivery of micro-amperage electrical stimulation. The electrical energy will pass from one strip to the other, which will contain the energy between the two metal electrodes.

A plurality of ultrasonic transducers 156 will be mounted on inner body tube 150, as seen in FIGS. 3, 4 and 6. The ultrasonic transducers 156 are commercially available products, and will be mounted to the inner body tube 150. The ultrasonic transducers 156 will be positioned to best correspond to the corpus cavernosum on either side of the penis.

In the embodiment shown in FIG. 3, there are 3 rows of ultrasonic transducers, alternating with three rows of Light Emitting Diodes (“LEDs”) 160. The light source used in embodiments of the present invention will be a Light Emitting Diode 160. The LEDs 160 will be mounted on a printed circuit board (“PCB”) 162, which includes circuitry so that the LEDs 160 are in electronic communication with the power source 50. The combination of LEDs 160 on PCB 162 forms the SMD light diode element, SMD referring to Surface-Mounted Diode.

The stimulatory device 100 will be closed by means of the rear base assembly 180. Rear base assembly 180 comprises a circular base 182, an anchor 190 for the combination line consisting of an electric line and a vacuum line, and a pair of gaskets. The base 182 includes a top surface 184 sized to receive a sealing ring 186. Base bottom 185 extends downward from base 182, and contains an opening 188 that receives inlet anchor 190, and which is positioned within opening 188 and notch 126. A second sealing gasket 192 is received within rear base assembly 180, and the threaded end of inlet anchor 190 is disposed between the second sealing gasket and the outer body tube (see FIGS. 2b, 4 and 6). Second sealing gasket 192 includes an extension 194 that fits within the inner ring 187 of the base 182. As shown in FIGS. 4-5, the inner body tube 150 will not make contact with the outer body tube 120 because the inner body tube 150 will be held in place by part of the outer rim 122 at the front, and the base 182 and second gasket 192 at the rear, and a void 200 is formed between the inner body tube 150 and outer body tube 120. It is through this void 200 through which a vacuum will be drawn, and air allowed to flow as the system is cycled on and off during treatment. The combination of the base 182 and the sealing ring 186 is made of a material having sufficient flexibility to permit the male stimulatory device 100 to form a seal around any penile size or configuration.

Depending upon the type of material used for the gaskets 136 and 192, the gaskets may require lubrication to maintain a good seal. When such lubrication is needed, a lubricant such as a petroleum jelly, or grease, could be used to lubricate the gaskets.

In parallel with the crystal transducers will be two metal strips. These metal strips will act as the active and dispersive pad for the delivery of micro-amperage electrical stimulation. The electrical energy will pass from one strip to the other, which will contain the energy between the two metal electrodes. LEDs configured to emit light at about 660 nm and 940 nm will be located in the top and bottom positions. Other embodiments may employ light within the range between about 660 nm and about 940 nm. In other embodiments, light within the range of about 350 nm to about 500 nm may be utilized. In some embodiments, the LEDs will be on at the same time as the ultrasound or electrical stimulation, which are located on the sides of the device. The LEDs will further improve blood flow and influence angiogenesis.

The process or method of use will begin with the penis being inserted into the chamber with vacuum being created. This vacuum may be a low pressure pulsed vacuum to simply stimulate the penis to an erection for the actual treatment. It will not be able to be set high enough to create a damaging excessive vacuum. A pressure release button 210 (FIGS. 1,6) will immediately break the vacuum in the event of an emergency, and also act as a redundant safety feature.

Once the penis is erect, the front cup 130 will be removed, and a quantity of ultrasound gel inserted into the stimulatory device 100 to fill it, after which the front cup 130 will be placed onto the outer body tube 120 to close it. The ultrasound gel enables the emitted ultrasound waves to electrical energy to make contact with the inserted penis and penetrate the tissue.

In an alternate method, the front cup 130 will be removed from the stimulatory device 100, the stimulatory device will be filled with a quantity of ultrasound gel sufficient to cover a user's penis, the cup 130 will be reattached, and the penis will be inserted into the stimulatory device 100.

The ultrasound energy will be delivered continually in what is clinically known as a stationery position. Normally, the transducer is moved to afford an even distribution of heat and to prevent depositing excessive energy into one treatment spot. As a result, the ultrasound energy will be delivered in a safe amount of wattage (about 0.5 W/cm²) and it will be pulsed to further ensure the proper amount of heat delivered to the corpus cavernosum. In embodiments, the pulsing will range from a duty cycle of about 1% to about 99%. Other embodiments may employ a narrower range of pulsing cycles. Yet another embodiment may start at a 15% duty cycle and ramp up slowly to a 75% duty cycle, and back down. The entire treatment will be 10 minutes and will be set to only allow one treatment a day. The best clinical results will come from these moderate treatments done daily and decreasing in regularity as vascularity improves. In one embodiment, the duty cycle will involve the application of vacuum, electrical stimulation, light stimulation and ultrasonic stimulation simultaneously. In other embodiments, the duty cycle may involve the use of one form of stimulation at a time, in a sequence, or may involve a combination of one or more forms of stimulation simultaneously.

As shown in FIG. 7, the treatment for the vagina will require the ultrasound, electrical stimulation, and LEDs to be placed on the outside of the tube and the insertion end of the device to be blocked off with the vacuum source emanating from that end rather than on top as with the penis device. Referring to FIG. 15, the stimulation points will be at the lower one third of the labia and slightly inserted into the first two inches of the vagina. This is where the majority of the sensory mechanisms are and the glands that secrete fluid for lubrication. By insertion of stimulatory device 300 partially into the vagina it allows the vacuum to be pulsed laterally, and physically exercise the muscles of the vagina.

FIGS. 7 and 8 are front and rear perspective views of the female stimulatory device 300, with FIG. 8 illustrating parts of the handle that are hidden from view in FIG. 7. FIGS. 11 and 12 are exploded views of the female stimulatory device 300, with FIG. 11 showing the components if seen from the front, and FIG. 12 showing a view from the rear; the main difference is that FIG. 12 shows parts of components which are hidden from view in FIG. 11, such as the reverse side of the handle, gasket or cap.

Referring to FIGS. 7-14, female stimulatory device 300 comprises a handle 310 which is generally hollow, that comprises one or more grips 312 on its outer surface, a strain relief 316 attached to handle cap 314, and a guard 320, the guard 320 being connected to the handle 310 by guard connector 318. The combination electric cord/vacuum line 72 is attached to the handle by means of the strain relief 316. The inside surface 322 of the guard 320 is sized to receive, and will receive a gasket 330, having a top side 332 and a bottom side 334. Bottom side 334 includes a plurality of electrical contacts 336 attached thereto. Forming the front-most part of the handle 310 is a handle cap 340 which includes a ring 342 that extends from cap 340's front surface 346 and as seen in FIGS. 13-14, supports the back end of inner tube 350 so it does not make contact with outer tube 370.

In construction somewhat similar to, but different from, the male stimulatory device 100, the female stimulatory device 300 includes an inside tubular element 350 onto which will be attached one or more metal strips 352 that will serve as electrodes for electrical stimulation, and a plurality of Light Emitting Diodes (“LEDs”) 360. The LEDs 360 will be mounted such that the emitted light will be directed outward from the tubular element 350.

An outer tubular element 370 will be open, and terminate in a front rim 372 onto which a cap 380 will be received by means of a press fit. In other embodiments, front rim 372 may comprise threads (not shown) and the front cap 380 may include a complementary surface so that the cap may be threaded onto the front rim 372. Outer tubular element 370 includes a plurality of perforations 374.

A plurality of ultrasonic transducers 156 are mounted in rows in openings 376 within the outer tubular element 370, and the transducers 156 are mounted so that the energy is directed outward from the tubular element 370. Tubular element 370 comprises a plurality of perforations 374, allowing for air flow during the application of the vacuum during the treatment process. The inner tube 350 is not in direct contact with the outer tubular element 370, and this orientation is seen more clearly in FIGS. 13-14, which are sectional views of the female embodiment 300. Front rim 372 acts to support the front of inner tube 350 which is partially inserted within cap 380 (FIG. 13). FIGS. 13-14 also show the insertion of combined cable 72 into the handle 300.

The female stimulatory device 300 will need to be lubricated prior to use, and this is done by distributing a quantity of ultrasound gel over the surface of the cap 380 and outer tubular element 370 sufficient to cover these surfaces. The ultrasound gel will be supplied either in a bulk quantity, or individual packets, as described below.

Internal wiring showing the electrical communications among the ultrasonic transducers, the electrodes, LEDs, and the handle are not shown; such electrical communication is known to those skilled in the art. The female stimulatory device is expected to be used with similar treatment times and frequency as that for the male stimulatory device, although some variations are to be expected. Activation of the power source 50 will start the treatment cycle for either device embodiment.

Without being bound by any scientific theory, it is believed that by combining various treatments that are known to affect tissue, the combined effect can contribute to the overall health of the penile and vaginal areas by stimulating cell growth, breaking up scar tissue, and increasing the flow of blood to the affected area.

In an alternate embodiment, systems of the present invention could be furnished in kit form. One kit embodiment could contain either the male or female stimulatory device, and a supply of ultrasound gel. Ultrasound gels, which are commercially available, could be supplied in bulk, such as an 8 ounce or 16 ounce container. Alternatives include syringes containing a specific quantity of gel for use with the male stimulatory device, for example only, 2-4 ounces, or packets with the smaller quantity of gel that would be needed for the female stimulatory device. Such a kit could also include replacement parts such as gaskets and gasket lubricant. The power supply 50 and the vacuum pump 70 are not intended for inclusion in any such kit of replacement parts, but could be included with a kit when the system is initially purchased.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A stimulatory device for use by males, the device comprising:
a tubular receptacle including an inner tube and an outer tube;
the outer tube having a first end that terminates in a rim, and a second end that terminates in a flange;
the inner tube comprising a plurality of perforations therethrough, a pair of electrodes, a plurality of ultrasound transducers, and a plurality of light sources, all attached to the surface of the inner tube and directing their energy to the inside of the inner tube; the inner tube positioned within the outer tube;
a cup, the cup sized to fit on and receiving the outer tube rim, the outer tube rim retaining the inner tube therein;
a rear base assembly, the rear base assembly comprising a base, the base including an inner ring, the inner ring sized to receive and receiving a gasket and an end of the inner tube, retaining the inner tube so the inner tube does not contact the outer tube;
a connection for a vacuum source; and
the light sources provide light at a wavelength ranging from about 660 nm to about 940 nm.

2. The male stimulatory device as described in claim 1, further comprising a gasket, the gasket positioned on the outer rim, between the outer tube and the cup.

3. The male stimulatory device as described in claim 1, further comprising a vacuum release mechanism, the vacuum release mechanism being positioned on the outside tube.

4. The male stimulatory device as described in claim 1, wherein the outside tube rim comprises threads, and the cap is threaded onto the outside tube.

5. The male stimulatory device as described in claim 1, wherein the rear base and the second gasket are flexible to seal the device around most penile sizes to provide a seal sufficient to maintain a vacuum when a vacuum is applied to the device.

6. A stimulatory device for females, the device comprising:
a handle, the handle comprising a handle member, a guard attached to the handle member, the guard having an forward surface sized to receive, and receiving a flange therein, and a handle cap, the flange being positioned between the guard and the handle cap;
an inner tube, the inner tube including a pair of electrodes attached thereto an outer tube the outer tube including a plurality of perforations therethrough;
a plurality of ultrasound transducers mounted on the outer tube, the outer tube having one end that terminates in a rim;
a plurality of light sources, the light sources attached to the inner tube;
a cap, the cap sized to receive and receiving the outer tube rim; and
a vacuum line connection, the vacuum line connection in communication with the handle, whereby when activated, the device employs a combination of vacuum, electrical stimulation, light stimulation and ultrasound stimulation to an area of a female human.

7. The device for females, wherein the handle further comprises a plurality of hand grips.

8. The device for females, wherein the inner tube is mounted on the outer tube rim and the handle cap ring, the inner tube positioned so it does not contact the outer tubular element.

9. The device for females as described in claim 6, wherein the light emitted has a wavelength ranging from about 640 nm to about 940 nm.
